(19) United States
(12) Patent Application Publication
(10) Pub. No.: US 2009/0250064 A1
(43) Pub. Date: Oct. 8, 2009

(54) Child Friendly Anesthesia Mask Attachment and Method of Use

(76) Inventor: Steffi Strawder, Clinton, MS (US)

Correspondence Address: FRASCOGNA COURTNEY, PLLC, P.O.BOX 23126, JACKSON, MS, 39225-3126, US

(21) Appl. No.: 12/098,685

(22) Filed: Apr. 7, 2008

(51) Int. Cl. A61M 16/06

(52) U.S. Cl. 128/207.11

(57) ABSTRACT

Children are often frightened by the bizarre appearance of standard anesthesia masks. A child friendly anesthesia mask attachment is disclosed, which covers some of portions of the standard anesthesia mask with child friendly objects. The child can then be encouraged to play with the mask and become familiar with placing it on his or her face prior to a medical procedure.

### FIELD OF THE INVENTION

This invention relates generally to anesthesia mask attachments and more specifically to anesthesia mask attachments that give the anesthesia mask a more comforting appearance to children.

### BACKGROUND OF THE INVENTION

When a patient undergoes a surgical or medical procedure, it is often necessary to cause the patient to inhale anesthetic gas for the purpose of numbing pain or sedating the patient for the duration of the procedure. Anesthetic gas is administered through the use of an industry standard anesthesia mask. These masks attach to a patients face with an air-tight seal that prevents the anesthetic gas from escaping the mask. This ensures the patient breathes only the mixture controlled by the anesthesia provider.

Anesthesia masks are very unattractive. Often patients, especially children, are afraid of these masks because of their bizarre appearance. Additionally, many patients fear the standard anesthesia masks because the patient perceives that the mask will interfere with the patient's breathing. Because of these fears, medical professionals often experience difficulty in convincing younger patients to wear the masks for proper anesthesia delivery.

Therefore, what is needed is a child friendly anesthesia mask attachment that reduces the effect of the aforementioned problems on the patient. This attachment should obscure the patient's view of some of the more frightening components of the standard anesthesia mask. The attachment should also be aesthetically pleasing to young patients so that the patient may become familiar with the mask and therefore less apprehensive about the mask's function. Additionally, the attachment should not impair the proper functionality of the standard anesthesia mask or hinder any medical professionals involved in the ensuing procedure. Furthermore, other desirable features and characteristics of the present invention will become apparent when this background of the invention is read in conjunction with the subsequent detailed description of the invention, appended claims, and the accompanying drawings.

### SUMMARY OF THE INVENTION

The present invention advantageously fills the aforementioned deficiencies by providing a child friendly anesthesia mask attachment that covers the standard anesthesia mask with animal noses, cartoon characters, or other objects that are comforting to a child.

In one particular embodiment of the present invention, a child friendly anesthesia mask attachment is placed over the primary gas tube of the standard anesthesia mask. The child is then allowed to play with the mask and attachment to become familiar with the mask's use. When the anesthesia is to be delivered, the attachment may be removed to allow the mask to be attached to an appropriate anesthesia delivery system.

In another embodiment of the present invention, the child friendly anesthesia mask attachment is constructed with two openings. A connecting tube is then inserted through both openings and connected to the primary gas tube. An anesthesia delivery system may then be attached to the primary gas tube of the mask via the connecting tube without the need of removing the child friendly anesthesia mask attachment.

The present invention now will be described more fully hereinafter with reference to the accompanying drawings, which are intended to be read in conjunction with both this summary, the detailed description and any preferred and/or particular embodiments specifically discussed. This invention may, however, be embodied in many different forms and should not be construed as limited to the embodiments set forth herein; rather, these embodiments are provided by way of illustration only and so that this disclosure will be thorough, complete and will fully convey the full scope of the invention to those skilled in the art.

### BRIEF DESCRIPTION OF THE DRAWINGS

The drawings contained herein exemplify three of the major embodiments of the claimed invention. It should be noted that the invention is not limited to the embodiments shown. The embodiments shown are purely examples, and the invention is capable of many variations of said embodiments. In the drawings,

FIG. 1 is an embodiment of a child friendly anesthesia mask attachment placed over the primary gas tube of a standard anesthesia mask.

FIG. 2 is a standard anesthesia mask and an embodiment of a child friendly anesthesia mask attachment being prepared for attachment to said anesthesia mask.

FIG. 3 is an embodiment of a child friendly anesthesia mask attachment as attached to a standard anesthesia mask and placed against a child's face.

FIG. 4 is an embodiment of the child friendly anesthesia mask attachment shown from the back, unconnected to an anesthesia mask, and including an attachment means.

FIG. 5 is a collection of minor embodiments of the child friendly anesthesia mask attachment featured together.

### DETAILED DESCRIPTION OF THE INVENTION

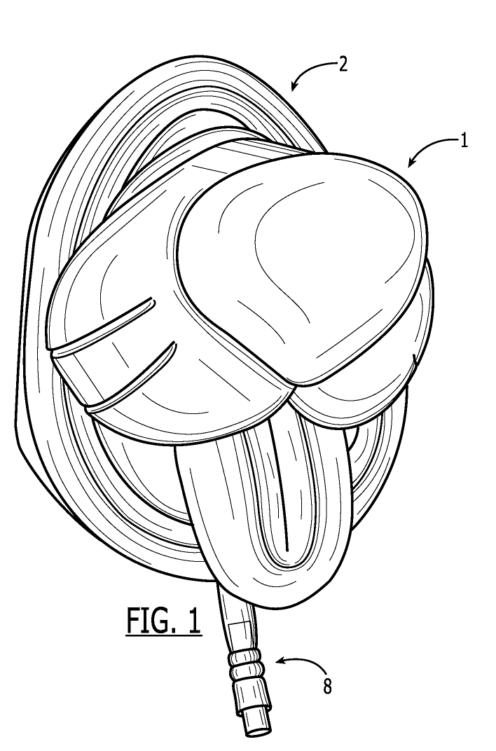

FIG. 1 illustrates a child friendly anesthesia mask attachment placed on a standard anesthesia mask 2. In this embodiment, the child friendly anesthesia mask attachment comprises a child attractive member 1 of a cartoon dog face. The child attractive member 1 performs the function of obscuring a patient's view of the standard anesthesia mask. While the present embodiment of the child attractive member 1 depicts a cartoon dog face, the disclosed invention encompasses any item which would distract a child from an impending surgery including but not limited to realistic animal faces, cartoon animal faces, animals, plants, toy automobiles, toy trains, toy farming equipment, toy airplanes, toy boats, seasonal icons, religious icons, food representations, cartoon characters, trading card characters, or video game characters. The preceding list is meant to be illustrative only and should not be construed as an exhaustive list of embodiments of a child attractive member 1. A child attractive member 1 may be any item that a child would perceive as a toy, is large enough to attract a child's attention, and light-weight enough to not interfere with the standard anesthesia mask's primary function. By way of example, the child attractive member 1 should not interfere with the function of the secondary 95 gas tube 8 of the standard anesthesia mask. The child attractive member 1 may be larger than the standard anesthesia mask 2 and may substantially extend from the mask as would be the case with an elephant trunk. However, the child attractive member 1 should be small enough that it does not interfere with the ability of medical personnel to perform their respective duties. The child attractive member 1 has no medical function other than to attract a patient's attention and encourage the patient to use the mask. The child attractive member 1 may also be treated with appropriate chemicals so that said child attractive member 1 emits a pleasing scent associated with the particular embodiment. Additionally, the child attractive member may be made of any material that would be consistent with the abovementioned criteria. However, in the preferred embodiment the child attractive member is not made of latex because of the possibility of allergic reactions by some patients. Also, the child attractive member may be made of transparent or semitransparent materials to allow medical personnel to view condensation forming on the inside of the mask and ensure that the patient is breathing properly.

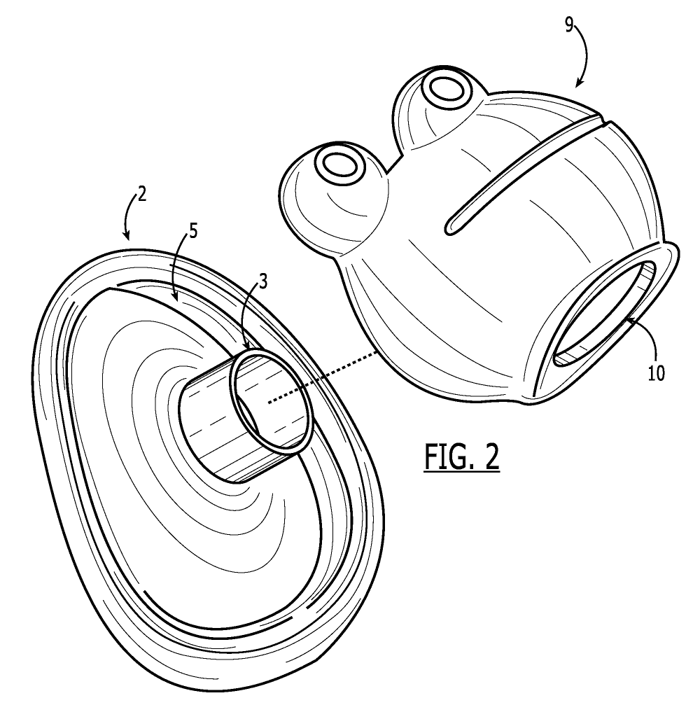

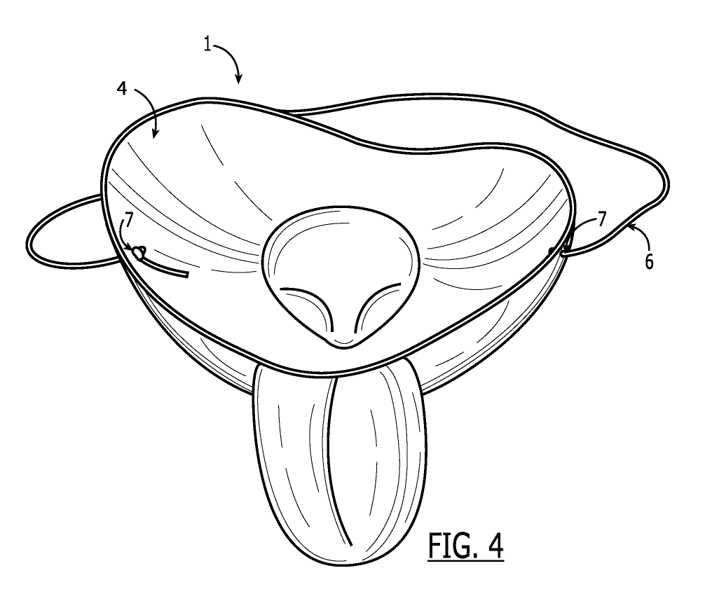

Briefly referencing FIG. 2, standard anesthesia masks have a primary gas tube 3. In the embodiment disclosed in FIG. 1, the child friendly anesthesia mask attachment further comprises a primary opening 4 in one surface of the child attractive member as depicted in FIG. 4. The primary opening 4 should be large enough to be placed over the primary gas tube 3 of the standard anesthesia mask 2. This allows a surface of the child friendly anesthesia mask attachment to rest on the front surface 5 of the standard anesthesia mask. The standard anesthesia mask 2 also typically comprises a secondary gas tube 8. The child friendly anesthesia mask attachment should be shaped so that it will not interfere with the function of the secondary gas tube 8.

The embodiment of the child friendly anesthesia mask attachment depicted in FIG. 4. also comprises an attachment means 6 which is attached to two attachment points 7 on the child attractive member 1. This attachment means 6 may be stretched around both the standard anesthesia mask 2 and the patient's head. In the preferred embodiment, the attachment means 6 is a strap made of elastic; however, the attachment means 6 may be any device which can temporarily attach the child attractive member 1 to a standard anesthesia mask 2. In this manner, the patient has the ability to wear and play with both the standard anesthesia mask 2 and the child friendly anesthesia mask attachment prior to surgery. Immediately prior to surgery, the attachment means 6 may be removed from the patient's head. Removing the attachment means allows the child friendly anesthesia mask attachment to be removed from the standard anesthesia mask 2. The primary gas tube 3 of the standard anesthesia mask 2 can then be connected to an appropriate anesthesia delivery system so that the medical procedure may commence.

As noted above, FIG. 2. illustrates a second embodiment of the child friendly anesthesia mask attachment and a standard anesthesia mask 2. In this embodiment, the child friendly anesthesia mask attachment may remain attached to the standard anesthesia mask 2 during surgery. The child friendly anesthesia mask attachment in this embodiment comprises a cartoon frog's head as the child attractive member 9. Any item that could function as a child attractive member 1 in the first embodiment could also function as a child attractive member 9 in the second embodiment. In this embodiment, the child friendly anesthesia mask attachment also comprises a secondary opening 10 in addition to the primary opening 4. The secondary opening 10 is present on a surface of the child attractive member 9. The primary opening 4 and the secondary opening 10 are connected by a space inside of the child attractive member 9.

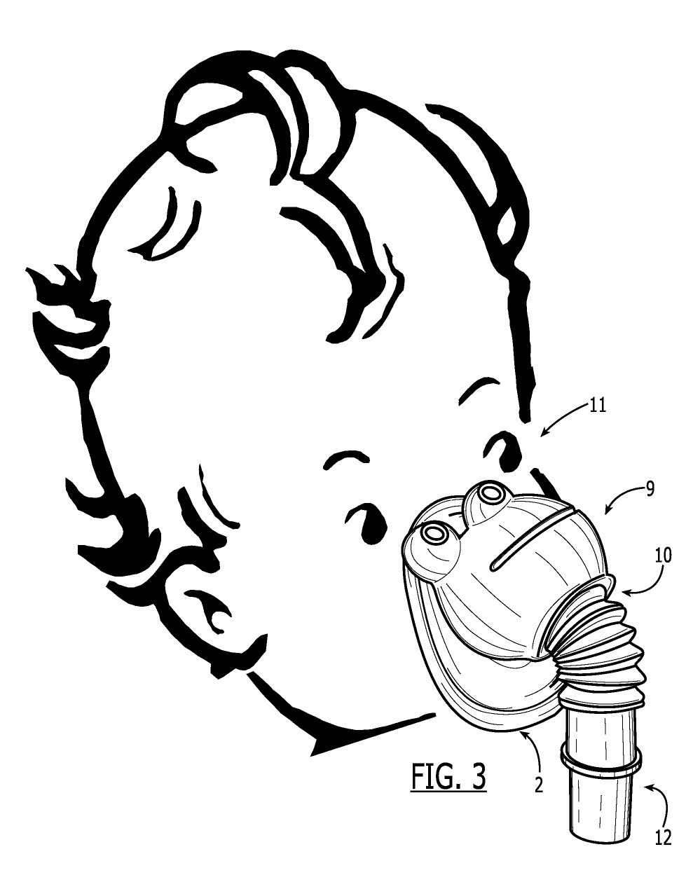

FIG. 3 illustrates the second embodiment of the child friendly anesthesia mask attachment connected to the standard anesthesia mask 2 and placed against a patient's face 11. To function during surgery, the standard anesthesia mask 2 must be attached to the pertinent anesthesia delivery system by a connecting tube 12. In order for the child friendly anesthesia mask attachment to remain on the mask during surgery, the connecting tube 12 is inserted through the secondary opening 10, the connecting space, and the primary opening 4. The connecting tube 12 can then be connected to the primary gas tube 3 of the standard anesthesia mask 2. In this embodiment, the primary opening 4, the secondary opening 10, and the connecting space must be large enough to accept the connecting tube 12 without constricting the gas flow. In the preferred embodiment, the primary opening 4 and secondary opening 10 should also be small enough to prevent the child friendly anesthesia mask attachment from sliding down the connecting tube 12. In this embodiment, the connecting tube 12 functions as an attachment means. Additional attachment means may be added for additional stability if desired.

In the second embodiment, the child attractive member 9 is not directly attached to the standard anesthesia mask 2. However, the child attractive member 9 is held in close proximity to the standard anesthesia mask 2 by the connecting tube 12 which functions as an attachment means. Close proximity is any distance at which a child would consider the child attractive member 9 to be a part of the standard anesthesia mask 2.

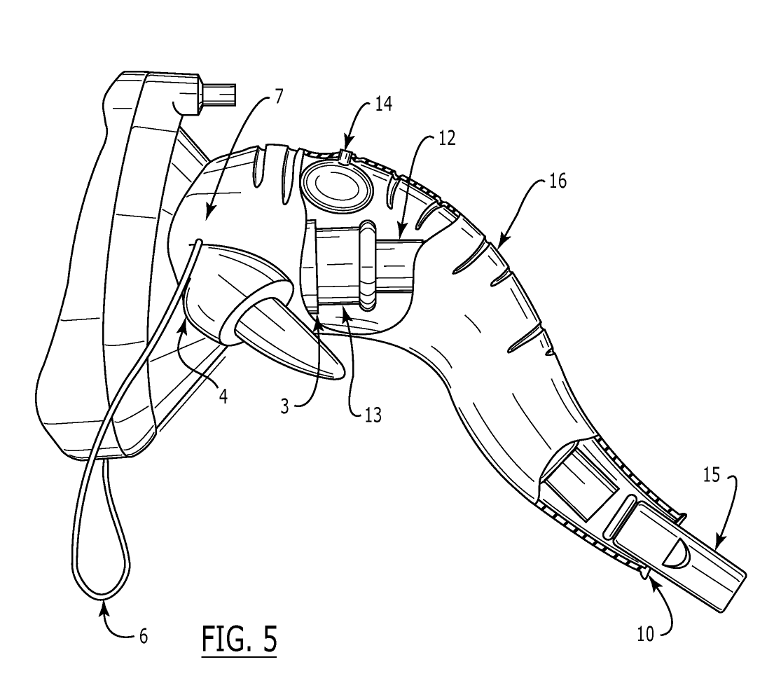

FIG. 5 is a collection of minor embodiments of the child friendly anesthesia mask attachment featured together. In this series of embodiments, the child friendly anesthesia mask attachment features an elephant trunk as the child attractive member 16. This embodiment features multiple attachment means 6 namely a strap attachment means 6 connected to two attachment points 7 and a connecting tube attachment means 12.

The present embodiment also features a universal tube attachment 13 connected to the connecting tube 12. The universal tube attachment 13 has an interior diameter of approximately eleven millimeters and an exterior diameter of approximately twenty two millimeters. The universal tube attachment 13 uses these dimensions because the primary gas tube 3 of a child size standard anesthesia mask may be inserted into the universal tube attachment 13 and the universal tube attachment 13 may also be inserted into the primary gas tube 3 of an adult size standard anesthesia mask. The presence of the universal tube attachment 13 attached to the connecting tube 12 ensures the child friendly anesthesia mask attachment is capable of connecting to any standard anesthesia mask. As with previous embodiments, the connecting tube 12 and universal tube attachment 13 are inserted into the space between primary opening 4 and the secondary opening 10.

The present embodiment also features a primary noise member 14. The primary noise member 14 functions in the same manner as an animal squeaky toy. When the user presses the portion of the child attractive member 16 containing the primary noise member 14, the primary noise member 14 will make a squeaking sound. The presence of the primary noise member 14 gives the patient one more item to play with and increases the likelihood that the patient will perceive the child friendly mask attachment as a toy and not as a medical device.

The present embodiment also features a secondary noise member 15. The secondary noise member 15 is a standard whistle. When the patient breathes into the standard anesthesia mask, the air will pass through the primary gas tube 3 of the anesthesia mask, into the universal tube attachment 13, through the connecting tube 12, and through the secondary noise member 15, causing the secondary noise member 15 to make a whistling sound. Like the primary noise member 14, the secondary noise member 15 may be included to increase the likelihood that the patient will perceive the child friendly mask attachment as a toy and not as a medical device. The secondary noise member 15 should be removably attached to the connecting tube so that the secondary noise member 15 may be removed before the connecting tube 12 is attached to an appropriate anesthesia delivery system.

It should be noted that the multiple features present in FIG. 5 are shown together only to decrease the number of drawings necessary in the present disclosure. Each of the features present in FIG. 5 may be used singly or in concert, in this or any previous or other embodiment. The presence of any feature in FIG. 5 is not intended and does not in any way require that feature to appear together with any other feature presented in FIG. 5. Any feature in FIG. 5 may be used or omitted from any given embodiment.

While the present invention has been described above in terms of specific embodiments, it is to be understood that the invention is not limited to these disclosed embodiments. Many modifications and other embodiments of the invention will come to mind of those skilled in the art to which this invention pertains, and which are intended to be and are covered by both this disclosure and the appended claims. It is indeed intended that the scope of the invention should be determined by proper interpretation and construction of the appended claims and their legal equivalents, as understood by those of skill in the art relying upon the disclosure in this specification and the attached drawings.

I claim:

1. A child friendly anesthesia mask attachment for attracting a patient's attention and encouraging a patient to breath from a standard anesthesia mask comprising:
a child attractive member further comprising a plurality of surfaces for attracting a patients attention and encouraging a patient to breath from a standard anesthesia mask; and
an attachment means for attaching the child attractive member to a standard anesthesia mask;
wherein a surface of the child attractive member is capable of being placed in contact with or in proximity to a standard anesthesia mask and held in place by the attachment means.

2. The child friendly anesthesia mask attachment of claim 1, wherein the attachment means further comprises a strap attached to the child attractive member and said attachment means is capable of being placed around a standard anesthesia mask.

3. The child friendly anesthesia mask attachment of claim 2, wherein the strap is capable of being placed around a standard anesthesia mask and a patient's head.

4. The child friendly anesthesia mask attachment of claim 1, wherein the attachment means further comprises an adhesive applied to a plurality of surfaces of the child attractive member.

5. The child friendly anesthesia mask attachment of claim 1, wherein the child attractive member is treated to emit a scent.

6. The child friendly anesthesia mask attachment of claim 1, wherein the child attractive member further comprises a primary noise member for making noise when pressed.

7. The child friendly anesthesia mask attachment of claim 1, wherein the child attractive member further comprises a secondary noise member for making noise when a patient breathes.

8. The child friendly anesthesia mask attachment of claim 1, wherein the child attractive member further comprises a primary opening.

9. The child friendly anesthesia mask attachment of claim 8, wherein the attachment means comprises the primary opening in the child attractive member.

10. The child friendly anesthesia mask attachment of claim 9, wherein the attachment means further comprises a universal tube attachment with an exterior diameter of approximately twenty-two millimeters and an interior diameter of approximately eleven millimeters.

11. The child friendly anesthesia mask attachment of claim 10, wherein
the universal tube attachment is inserted into the primary opening of the child attractive member.

12. The child friendly anesthesia mask attachment of claim 9 wherein, the attachment means further comprises a secondary opening connected to the primary opening by a space.

13. The child friendly anesthesia mask attachment of claim 12 wherein, the attachment means further comprises a connecting tube.

14. The child friendly anesthesia mask attachment of claim 13 wherein, the connecting tube is inserted through the primary opening, the connecting space, and the secondary opening, and is capable of being attached to a standard anesthesia mask.

15. The method of using a child friendly anesthesia mask attachment for attracting a patient's attention and encouraging a patient to breath from a standard anesthesia mask comprising;
obtaining a child friendly anesthesia mask attachment further comprising a child attractive member and an attachment means;
obtaining a standard anesthesia mask further comprising a plurality of surfaces;
placing the child attractive member against a surface of a standard anesthesia mask;
attaching the child attractive member to the standard anesthesia mask with the attachment means; and
giving the standard anesthesia mask with the attached child attractive member to a patient.

16. The method of using a child friendly anesthesia mask attachment for attracting a patient's attention and encouraging a patient to breath from a standard anesthesia mask comprising;
obtaining a child friendly anesthesia mask attachment comprising a connecting tube and a child attractive member further comprising a primary opening, a secondary opening, and a connecting space;
obtaining a standard anesthesia mask comprising a primary gas tube;
placing the connecting tube of the child friendly anesthesia mask attachment through the primary opening, connecting space, and secondary opening of the child attractive member of the child friendly anesthesia mask attachment,
attaching the connecting tube of the child friendly anesthesia mask attachment to the primary gas tube of the standard anesthesia mask; and giving standard anesthesia mask with the attached child friendly anesthesia mask attachment to a patient.
